(19) United States
(12) Patent Application Publication
(10) Pub. No.: US 2020/0289042 A1
(43) Pub. Date: Sep. 17, 2020

(54) Systems, Devices, and Methods of Determining Data Associated with a Persons Eyes

(71) Applicant: Eyelab, LLC, Austin, TX (US)

(72) Inventors: Michael Patton, Austin, TX (US); Gary Lickovitch, Alpharetta, GA (US); Tanaya Meaders, Monroe, GA (US)

(73) Assignee: Eyelab, LLC, Austin, TX (US)

(21) Appl. No.: 16/818,937

(22) Filed: Mar. 13, 2020

Related U.S. Application Data
(60) Provisional application No. 62/818,028, filed on Mar. 13, 2019.

(51) Int. Cl. A61B 5/16; G06F 3/01; G06K 9/00; G06F 3/14; A61B 3/113; A61B 5/00; A61B 3/00

(52) U.S. Cl. CPC A61B 5/163; G06F 3/013; G06K 9/0061; G06F 3/14; A61B 3/113; A61B 3/14; A61B 5/746; A61B 3/0091; A61B 5/6803; A61B 5/6898; A61B 5/7282; A61B 5/742

(57) ABSTRACT

A system may detect a neurological impairment of a patient based, at least in part, on optical data. The system may include a computing device including a display to present visual information to a patient and an optical sensor to capture optical data of eyes and facial muscles surround the eyes of the patient. The computing device may further include a processor to generate data indicative of impairment based on the optical data.

### CROSS-REFERENCE TO RELATED APPLICATION(S)

The present disclosure is a non-provisional of and claims priority to U.S. Provisional Patent Application No. 62/818,028 filed on Mar. 13, 2019 and entitled “Physiological State Evaluation Devices, Systems, and Methods”, which is incorporated herein by reference in its entirety.

### FIELD

The present disclosure is generally related to physiological state evaluation devices, systems, and methods, and more particularly, to devices, systems, and methods configured to capture data (such as optical data, pressure data, vibration data, other data, or any combination thereof) associated with a person's eyes and the muscles around the person's eyes and to determine one or more physiological states based on the captured data.

### BACKGROUND

A variety of factors may adversely impact cognitive processes and associated performance of a person, both in sports and in other aspects of life. For example, head injuries, genetic influences, disease/infection, exposure to toxic substances, and lifestyle factors (e.g., drugs use, alcohol use, dehydration), other factors, or any combination thereof may adversely impact a physiological state of a person, interfering with cognitive function and adversely affecting a person's life, including the person's well-being, performance, and even the person's life span. For example, lifestyle factors may adversely impact executive functions in cognition, such as visuo-spatial processing. In some instances, physiological state changes may interfere with the way neurons send, receive, and process signals by inhibiting neural pathways.

Similarly, head injuries or traumatic brain injuries, such as a concussion, may adversely impact the person's physiological state, such as by negatively affecting the person's short-term memory, reaction time, eye movements, behaviors, moods, pupillary reflexes, and other physiological functions. A concussion is a type of traumatic brain injury that may be caused by a bump, blow, or jolt to the head or by an impact that causes the head and brain to move rapidly back and forth. For example, falls, vehicular crashes, bicycle crashes, assaults, and sports impacts can cause concussions. Such impacts can cause the brain to bounce around or turn in the skull, causing bruising and stretching of brain tissue compromising brain cells, creating chemical changes in the brain, cognitive impairments, or any combination thereof. Some head injuries may also cause the brain to swell. Such bruising, stretching, or swelling of brain tissue may impair the person's physiological state.

### SUMMARY

Embodiments of testing devices, systems, and methods are described below that can capture data associated with a person's eyes and surrounding eye muscles to detect one or more parameters indicative of physiological state changes. Such physiological state changes may be representative of brain injury, impairment, dehydration, or any combination thereof. In some implementations, a device may present visual data to a display and may capture image data associated with a person's eyes and eye muscles as the person looks at and tracks the visual data. The captured image data may be processed by the device or by an associated computing device (communicatively coupled to the device) to determine one or more parameters indicative of physiological state changes, which may be representative of cognitive impairment, brain injury, impairment, dehydration, or any combination thereof based on the image data.

In some implementations, a system may detect physiological state changes representative of cognitive impairment of a person based, at least in part, on optical data. The system may include a computing device including a display to present visual information to a person and an optical sensor to capture optical data of eyes, optical data associated with facial muscles around the eyes of the person, other data, or any combination thereof. The computing device may further include a processor to generate data indicative of impairment based on the optical data.

In some implementations, a system may include a computing device. The computing device may include one or more sensors to capture data associated with a person's eyes as the person observes one or more objects moving in a three-dimensional space. The computing device may include a display to present information related to the captured data.

In other implementations,

In still other implementations, a system may include a computing device. The computing device may include one or more sensors to capture data associated with a person's eyes as the person observes one or more objects moving in a three-dimensional space. The computing device may also include a processor coupled to the one or more sensors and configured to generate information related to the capture data and a display coupled to the processor and configured to present the generated information.

### BRIEF DESCRIPTION OF THE DRAWINGS

FIG. 1 depicts a diagram of systems and devices to provide a physiological state evaluation, in accordance with certain embodiments of the present disclosure.

FIG. 2 depicts a flow diagram of a process of determining data indicative of a person's physiological state, in accordance with certain embodiments of the present disclosure.

FIG. 3 depicts a block diagram of a system including an analytics system to provide physiological state evaluation and analysis, in accordance with certain embodiments of the present disclosure.

FIG. 4 depicts a block diagram of a computing device, in accordance with certain embodiments of the present disclosure.

FIG. 5 depicts a block diagram of a computing device such as a virtual reality device or a smart glasses device, in accordance with certain embodiments of the present disclosure.

FIG. 6 depicts a diagram of optical test data that can be presented on one of the computing devices of FIGS. 4 and 5, in accordance with certain embodiments of the present disclosure.

FIG. 7 depicts a diagram of an eye-tracking test that uses three-dimensional movement, in accordance with certain embodiments of the present disclosure.

FIGS. 8A-8C depict view angles that may be used to determine impairment, in accordance with certain embodiments of the present disclosure.

FIG. 9 depicts a system to capture optical data of a person as the person observes a three-dimensional moving object, in accordance with certain embodiments of the present disclosure.

FIG. 10 depicts an image including an image processing matrix and including elements or areas for analysis, in accordance with certain embodiments of the present disclosure.

FIG. 11 depicts a flow diagram of a method of determining impairment based on optical data, in accordance with certain embodiments of the present disclosure.

FIG. 12 depicts a flow diagram of a method of determining impairment based on optically detected ocular pressure, in accordance with certain embodiments of the present disclosure.

FIG. 13 depicts a flow diagram of a method of determining impairment based on motion and orientation data, in accordance with certain embodiments of the present disclosure.

In the following discussion, the same reference numbers are used in the various embodiments to indicate the same or similar elements.

### DETAILED DESCRIPTION OF ILLUSTRATIVE EMBODIMENTS

Embodiments of systems, methods, and devices are described below that may capture data associated with a person's eyes, facial area surrounding the person's eyes, other data, or any combination thereof, and may automatically detect a change in physiological state, indicative of impairment based on the captured data. The captured data may include optical data, pressure data, vibration data, other data, or any combination thereof.

Examples of cognitive disorders that manifest with cognitive impairment disturbances may include, but are not limited to, a head injury, concussion or other traumatic brain injury; a chemical impairment (such as due to consumption of alcohol or illicit drugs, abuse of prescription drugs, smoking marijuana, allergic reaction, other sources of chemical impairments, exposure to toxic substances, or any combination thereof); early indicators of neurocognitive diseases or infections (such as Multiple Sclerosis, Parkinson's disease, Meningitis, AIDS related dementia, or any combination thereof); genetic influences (such as Alzheimer's disease); strokes; dementia; lifestyle factors (such as malnutrition, poor diet, dehydration, overheating—increased core body temp, or any combination thereof); other cognitive disorders or impairments; or any combination thereof.

In some implementations, an electronic device may be worn by a person. For example, the electronic device may include a virtual reality (VR) headset device, a smart glasses device, a smartphone positioned in front of the user's eyes, or another electronic device. The electronic device may include a display to provide data (such as moving images, colors, texts, light of varying intensities, other information, or any combination thereof). At the same time, the electronic device may capture optical data associated with a person's eyes, including facial muscles, skin surrounding the person's eyes, other data, or any combination thereof as the person observes the data on the display. The optical data may be used to determine physiological state changes, which may be indicative of cognitive impairment of the person. The optical data may be processed by the electronic device or may be communicated to a computing device coupled to the electronic device by a wired or wireless communications link so that the computing device can process the optical data.

In some implementations, the optical data may provide a biometric fingerprint that can be used to uniquely identify the person based, for example, on images of the user's eye. Further, the optical data may include color variations that may be imperceptible to the human eye, but which may reveal blood flow within and around the person's eyes. Additionally, the optical data may include data variations that can reveal details of the person's pupil reflexes, eye movements (smooth pursuits, saccadic movements, irregular, convergent, divergent, and so on), reaction time, eye shape, facial muscle movements, other information, or any combination thereof In some implementations, the optical data may also reveal ocular pressure based on movement data of the eye and the facial muscles, for example, in response to a physical impulse or vibration.

In some implementations, one or more transducers may be included in the electronic device. In one possible implementation, a transducer may be responsive to an electronic signal to apply a physical vibration or impulse to the person's skin, such as the skin below the user's eyes, and the optical data may observe eye and facial movements in response to the vibration or impulse. In some instances, the device or a computing device may infer swelling or ocular pressure based on the eye movements, facial movements, other data, or any combination thereof in response to the vibration or impulse. Other implementations are also possible.

In some implementations, as the person observes visual data on the display, optical data may be captured of the person's retina and optionally the interior of the person's eye through the pupil. The optical data may be used to detect macular degeneration, glaucoma, bulging eyes (swelling), cataracts, cytomegalovirus (CMV) retinitis, crossed eyes (or strabismus), macular edema, possible or impending retinal detachment, an irregular shaped cornea, lazy eye, ocular hypertension, uveitis, other ocular conditions, or any combination thereof.

In some implementations, the electronic device may include orientation and motion sensors, which may generate signals proportional to the movement and stability of the person. For example, a person with a neurocognitive impairment condition may sway or otherwise have difficulty standing still and straight without tilting. The orientation and motion sensors may generate signals representative of dizziness or changes in balance of the person, which signals may be indicative of physiological state changes representative of cognitive impairment. Other implementations are also possible.

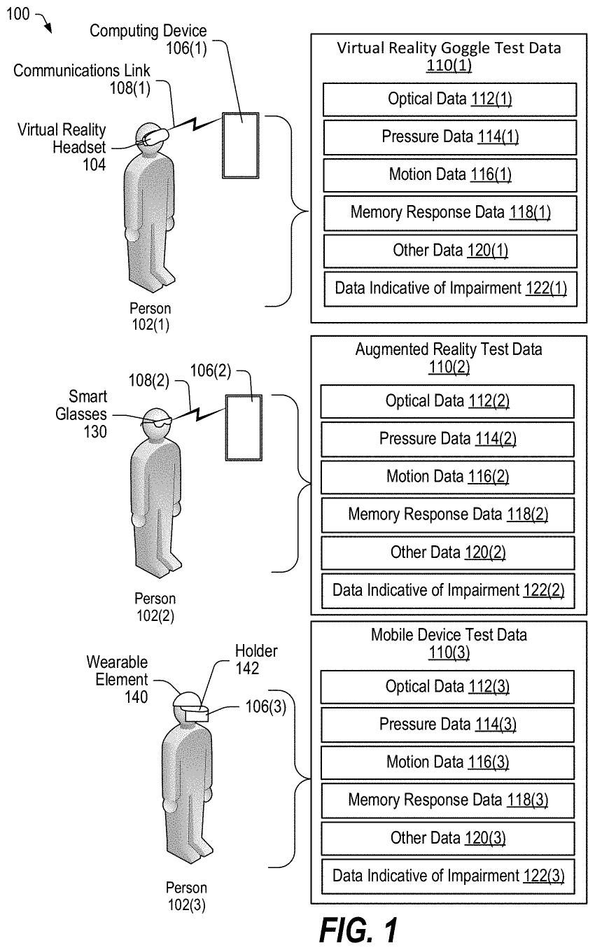

It should be understood that the systems, devices, and methods may be implemented in a variety of configurations. In one implementation, a device may be self-contained and configured to display images, capture data, and determine physiological changes based on the captured data. In another implementation, the device may display images, capture data, and communicate the captured data to a computing device (through a wired or wireless connection), and the computing device may determine physiological changes based on the captured data. In still other implementations, the computing device may communicate with another computing device (such as a computer server) through a network to compare at least a portion of the captured data to previously captured data associated with the person. The previously captured data may include baseline physiological data that can be used as a basis for comparison to detect changes, which may be the result of an impact or other condition. In some instances, deviation from a baseline may be indicative of a physiological change, which may be used as a basis for diagnosis, such as to determine whether a person should enter a concussion protocol. Examples of implementations are described below with respect to FIG. 1.

FIG. 1 depicts a diagram 100 of systems and devices to provide physiological state evaluations, in accordance with certain embodiments of the present disclosure. The diagram 100 depicts a first person 102(1) wearing a virtual reality (VR) headset 104, which may communicate with a computing device 106(1) through a communications link 108(1). The computing device 106(1) may be a tablet computer, a smartphone, a laptop computer, another computing device, or any combination thereof. The communications link 108(1) may be a wired communications link (such as a Universal Serial Bus (USB) connection or another wired connection), a radio frequency (RF) communications link (such as a Bluetooth® communications link, a Wi-Fi® communications link, an 802.11x IEEE communications link, another RF communications link), or any combination thereof.

The VR headset 104 may include a display and a plurality of sensors, including optical sensors (such as a camera). The display may present visual data for viewing by the person. For example, the VR headset 104 may present images, objects, colors, different brightness intensities, information, or any combination thereof to the display. In some implementations, the VR headset 104 may present a moving object on the display such that the moving object appears to move three-dimensionally (away from and toward the user as well as side to side). For example, the VR headset 104 may present an object that appears to move from a distance at a center of a field of view directly toward a point between the person's eyes (i.e., a convergence test).

Optical sensors of the VR headset 104 may concurrently capture optical data 110(1) associated with the eyes, facial area surrounding the eyes of the person, or any combination thereof as the person observes the visual data on the display. In the example of the convergence test, the optical data 110(1) may capture divergence of the person's eyes as the object appears to move toward the person. The optical data 110(1) may also include facial muscular movements, eye movements (rapid movement, tracking movement, and so on), pupil reflexes, pupil shape, blood flow, eye shape, swelling information, divergence data, biometric data, miniscule color variations, other optical information, or any combination thereof. Such optical data may be too rapid or too small to be detected by the naked eye of the doctor or observer, but changes may be amplified by the system to provide a readily discernable physiological response.

The VR headset 104 may also include one or more transducers to apply a vibration or impulse to the person's face while the optical sensors capture the optical data. For example, the transducers may impart a vibration or impulse that may cause the person's face and eyes to undulate, providing movements that can be captured in the optical data 110(1) from the optical sensors. Optical data 110(1) of the undulations may be used to infer pressure data 112. In an example, when a person has facial swelling, the vibrations may be dampened by the pressure more rapidly than when such swelling is not present.

The plurality of sensors may also include orientation sensors, motion sensors, gyroscopes, other sensors, or any combination thereof. For example, as the person wears the VR headset 104, the sensors may generate electrical signals proportional to movements of the person, which signals may represent motion data 114(1). In some implementations, swaying movements when the user is standing still may differ from person to person; however, variations in a person's movements relative to baseline tests may be indicative of traumatic brain injury.

Further, in some implementations, the VR headset 104 may present information to the person, such as a list of words, an arrangement of objects, objects of different colors, and so on, and may instruct the person to memorize the information. Then, the VR headset 104 may present visual data and may monitor the eyes, facial area surrounding the eyes, or any combination thereof as the person observes the visual data. After presenting the visual data, the VR headset 104 may test the person's recall of the information to determine memory response data 116(1).

In some implementations, the VR headset 104 may capture other data 118(1). The other data 118(1) may include differences between the measurement data and one or more baseline measurements. The other data 118(1) can also include retinal data and other information.

In some implementations, the VR headset 104 or the computing device 106(1) may include a processor configured to analyze the optical data 110(1), pressure data 112, motion data 114(1), memory response data 116(1), other data 118(1), or any combination thereof to detect impairment and to produce data indicative of impairment 120(1). For example, the processor may analyze the optical data 110(1) to determine biometric data, which can be used to uniquely identify the person. Further, the processor may present data to the display and receive optical data 110(1) as the person watches the data on the display. The processor may analyze the optical data 110(1) to detect facial muscle movements, eye movements (rapid movements, tracking movements (smooth or otherwise), divergence, other eye movements, or any combination thereof), pupil reflexes, pupil shape, eye shape, swelling, retinal injury, and so on. The processor may further analyze the motion data 114(1) to detect movement indicative of dizziness or imbalance. The processor may determine data indicative of impairment 120(1) of the person 102(1) based on the optical data 110(1), the pressure data 112, the motion data 114(1), the memory response data 116(1), other data 118(1), or any combination thereof.

While a VR headset 104 may provide a self-contained testing apparatus for determining physiological state changes representative of cognitive impairment of a person 102(1), it may also be possible to provide similar testing, obtain similar optical data 110, and determine data indicative of impairment 120 using other devices. For example, smart glasses 130 may be worn by a person 102(2) and may communicate with a computing device 106(2) through a communications link 108(2). The smart glasses 130 may be configured to present visual data to a display. The visual data may include objects that move, various colors, various intensities of light, and other data. In some implementations, the smart glasses 130 may present an augmented reality, such as by presenting the objects superimposed over visual objects in the real world.

The smart glasses 130 may include one or more optical sensors to capture optical data 110(2) of the person 102(2). The smart glasses 130 may also include one or more motion sensors (such as an inertial measurement unit (IMU) sensor) to generation motion data 114(2). Further, the smart glasses 130 may present information to the display and instruct the person to remember the information. Subsequently, the smart glasses 130 may test the person's memory with respect to the information to determine memory response data 118(2). The smart glasses 130 may also produce other data 118(2). In some implementations, a processor of the smart glasses 130 or a processor of the computing device 106(2) may analyze the data to determine data indicative of impairment 120(2).

In another implementations, a device may include a wearable element 140 including a holder 142 configured to secure the computing device 106(3) in front of the person's eyes. For example, the wearable element 140 may include a cap, and the holder 142 may extend from the cap and secure the computing device 106(3) at a pre-determined distance from the person's eyes. The wearable element 140, the holder 142, or both may be adjustable to fit the person 102(3) and to present the computing device 106(3) at a selected distance from the person's eyes. In this example, the computing device 106(3) may be a smartphone, a tablet computer, or other computing device with a display and sensors.

The computing device 106(3) may present visual information to the person, and may capture optical data 110(3), motion data 114(3), memory response data 116(3), and other data 118(3). A processor of the computing device 106(3) may analyze the optical data 110(3), motion data 114(3), memory response data 116(3), and other data 118(3) to determine data indicative of impairment 120(3).

The data indicative of impairment 120 may be determined, for example, by comparing captured data to one or more thresholds. In some implementations, the thresholds may be determined by analyzing data collected from a plurality of persons 102. Over time, a generalized average baseline measurement may be determined that may be used to determine impairment. Such impairments may include traumatic brain injuries (e.g., a concussion), chemical impairments or exposure to toxic substances, neurological diseases or infections, lifestyle factors, eye injuries, and so on. The processor may compare the captured data to the baseline and may determine impairment when the captured data deviates from the baseline by more than a threshold amount. Other implementations are also possible.

In some implementations, a person 102 may be initially tested, such as prior to injury, one or more times to determine a baseline for the person 102. In some implementations, such data may be used to determine a biometric signature for the person 102. The baseline may be associated with the biometric signature in a database, which may be stored on the device or on a server accessible through a computing network, such as the Internet. Subsequently, when the person 102 is tested, a biometric signature may be determined from the optical data. The biometric signature may be used to retrieve the baseline for the person 102, and the captured data may be compared to the baseline to determine impairment when the captured data deviates from the baseline by more than a threshold amount. Other implementations are also possible.

The computing device 106 may retrieve the baseline for the person 102 from a local memory of the computing device 106, from a memory of another computing device 106, from a database accessible through a communications network (such as the Internet), or any combination thereof.

Various impairments may be determined based on the captured data. Such impairments can include head injury or traumatic brain injuries (such as concussions), chemical impairments (such as alcohol or drugs), injuries (such as retinal detachments), dehydration, other impairments, or any combination thereof.

It should be understood that a cognitive impairment (CI) includes a situation in which a person has trouble remembering, learning new things, concentrating, or making decisions. CI may not be caused by any specific disease/condition and is not necessarily limited to a specific age group; however, Alzheimer's disease, other dementias, Parkinson's disease, stroke, fatigue, traumatic brain injury, developmental disabilities, and other conditions may manifest as CI. Common signs of CI can include memory loss, change in mood or behavior, vision problems, trouble exercising judgment, and so on. The DSM-5 (Diagnostic and Statistical Manual of Mental Disorders) now lists cognitive disorders as neurocognitive disorders indicating that there is some type of involvement of the brain.

Embodiments of the systems, devices, and methods described herein may provide optical data consistent with one or more cognitive evaluations (such as moving objects, item to be memorized, and so on) to a display. The systems, devices, and methods may capture optical data of the person's eyes and face as the person observes the data presented on the display. Such video data may be processed to detect a physiological state of the person. The physiological state may include physical conditions (e.g., dehydration, detached retina, swelling, and so on) and which may include CIs or neurological disorders. Some categories of types of CIs may include 1) Genetic Influences (such as Alzheimer's disease, Parkinson's disease, stroke, dementia, and so on); 2) Head Injury (such as a closed head injury, traumatic brain injuries (concussions, contusions, and so on), other head injuries, or any combination thereof); 3) Disease/Infection (such as Meningitis (from virus), Multiple Sclerosis (Autoimmune and attacks myelin), Parkinson's disease (dopamine producing cells die), AIDS (dementia from virus), Macular Degeneration, Retinal Detachment, other conditions, or any combination thereof); 4) Exposure to Toxic Substances (such as neurotoxins (lead, heavy metals, paint fumes, gasoline, aerosol), alcohol, drugs (legal and illegal), other toxins, or any combination thereof); and 5) Lifestyle Factors (such as malnutrition, dehydration, overheating (core body temperature, over exertion, etc.), other factors, or any combination thereof.

In one possible implementation, dehydration of a person may manifest as a physiological change that can be determined from the captured optical data. Symptoms may include feelings of confusion or lethargy, lack of urination for an extended period (such as for eight hours), rapid heartbeat, low blood pressure, weak pulse, inability to sweat, sunken eyes, and so on. In some instances, dehydration may also manifest as eye strain. Decreased lubrication and absence of tear production, tired eyes, blurred vision, headaches, and double vision are all symptoms of eye strain. Other optically detectable symptoms of dehydration are also possible, such as a change in skin elasticity relative to a baseline. In some implementations, the systems, devices, and methods may determine a change in skin elasticity relative to a baseline based on eye movements (and optionally damping of vibrations).

Dehydration can cause shrinkage of brain tissue and an associated increase in ventricular volume. The increase in BOLD (blood oxygen level dependent) response after dehydration suggested an inefficient use of brain metabolic activity. This pattern may indicate that participants may have exerted a higher level of neuronal activity in order to achieve an expected performance level. Given the limited availability of brain metabolic resources, these findings suggest that prolonged states of reduced water intake may adversely impact executive functions, such as visual-spatial processing which may include the ability to represent and mentally manipulate three-dimensional objects. Overheating may encompass dehydration and may have similar physiological manifestations. Other physiological states and other determinations may be made based on the video data, depending on the implementation. The systems, devices, and methods may determine the person's level of dehydration based on deviation of the persona's responsiveness relative to the baseline.

If a person is dehydrated or in a compromised state of hydration, the systems, methods, and devices may detect a physiological state that includes a change in rapid eye tracking of three-dimensional movement. The change may be relative to a standard baseline or relative to a baseline corresponding to the person. The baseline may be determined from a local memory of the computing device 106, from another computing device 106, from a database accessible through a communications network (such as the Internet), from another source, or any combination thereof.

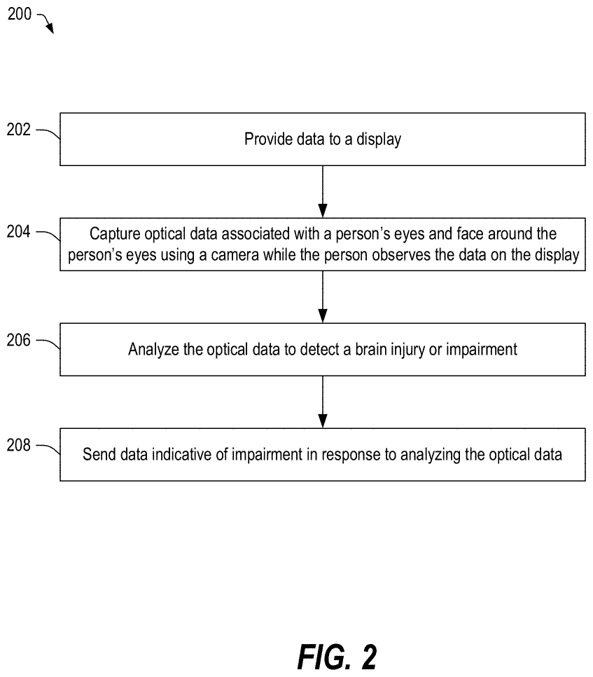

FIG. 2 depicts a flow diagram of a process 200 of determining data indicative of a person's physiological state, in accordance with certain embodiments of the present disclosure. At 202, data may be provided to a display. For example, visual data may be presented on a display of a VR headset 102, smart glasses 130, or a mobile computing device 106. The data may include moving objects, information, varying colors, varying intensities of light and dark, other visual elements, or any combination thereof.

At 204, optical data associated with a person's eyes and face around the person's eyes may be captured using a camera (or other optical sensor) while the person observes the data on the display. For example, one or more optical sensors integrated with the display device may capture optical data while the person observes the data on the display. In some implementations, other types of sensors may also be used.

At 206, the optical data may be analyzed to identify physiological state changes representative of cognitive impairment or brain injury. For example, eye movements, divergence, pupil reflexes, pupil shape, eye shape, minute color changes, minute shape changes, facial movements, other data, or any combination thereof may be analyzed to detect information indicative of impairment. In a particular example, presentation of an object moving from far away toward a point between the user's eyes can be presented on the display, and divergence of the user's eyes can be determined to detect impairment. In another particular example, pupillary reflexes, a rate of change of the pupil size, variations in the pupil shape over time, or other measurements may be indicative of CI. Additionally, irregular or non-smooth eye movements may be indicative of CI. Other examples are also possible.

At 208, data indicative of impairment may be sent in response to analysis of the optical data. In some implementations, the data indicative of impairment may be presented on a display of a computing device, such as a smartphone. In an example, the data indicative of impairment may include an email or a graphical interface, which may be sent to a computing device 106 or to another device. In some implementations, the data indicative of impairment may include an indication of the impairment and a basis for the determination, which may allow a physician to review the information. The data indicative of impairment may include the optical data (including, for example, magnification of selected pixels or subsets of image data values). Other implementations are also possible.

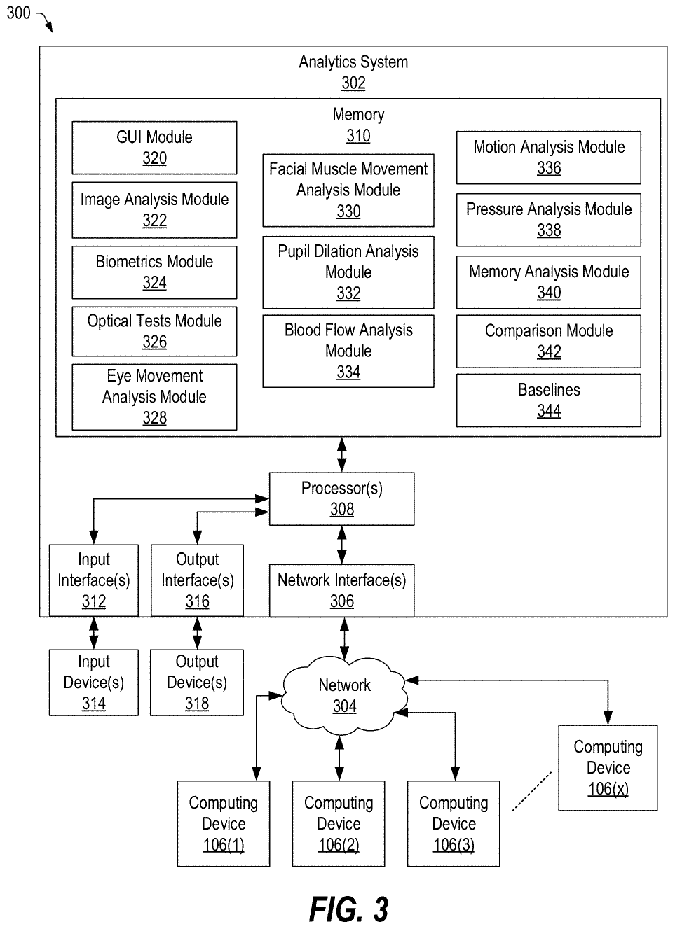

FIG. 3 depicts a block diagram of a system 300 including an analytics system 302 to provide neurological testing and analysis, in accordance with certain embodiments of the present disclosure. That analytics system 302 may be communicatively coupled to one or more computing devices 106 through a network 304. The network 304 may include local area networks, wide area networks (such as the Internet), communication networks (cellular, digital, or satellite), or any combination thereof.

The analytics system 302 may include one or more network interfaces 306 configured to communicate with the network 304. The analytics system 302 may further include one or more processors 308 coupled to the one or more network interfaces 306. The analytics system 302 may include a memory 310 coupled to the processor 308. The analytics system 302 may include one or more input interfaces 312 coupled to the processor 308 and coupled to one or more input devices 314 accessible by an operator to provide input data. The input devices 314 may include a keyboard, a mouse (pointer or stylus), a touchscreen, a microphone, a scanner, another input device, or any combination thereof. The analytics system 302 may also include one or more output interfaces 316 coupled to the processor 308 and coupled to one or more output devices 318 to display data to the operator. The output devices 318 may include a printer, a display (such as a touchscreen), a speaker, another output device, or any combination thereof

The memory 310 may include a non-volatile memory, such as a hard disc drive, a solid-state hard drive, another non-volatile memory, or any combination thereof. The memory 310 may store data and processor-executable instructions that may cause the processor 308 to analyze optical data and other data and to determine data indicative of impairment 120 for a person 102. The memory 310 may include a graphical user interface (GUI) module 320 that may cause the processor 308 to generate a graphical interface including text, images, and other items and including selectable options, such as pull-down menus, clickable links, checkboxes, radio buttons, text fields, other selectable elements, or any combination thereof. The processor 308 may send the graphical interface to the output device 318, to one or more of the computing devices 106, or any combination thereof.

The memory 310 may further include an image analysis module 322 that may cause the processor 308 to receive image data from one or more of the computing devices 106. The image analysis module 322 may cause the processor 308 to selectively process image values from the image data. For example, the image analysis module 322 may cause the processor 308 to analyze pixel color variations over time and to analyze other image data to determine various parameters. Further, the image analysis module 322 may cause the processor 308 to determine swelling, eye measurements, and other data. Other implementations are also possible.

The memory 310 can also include a biometrics module 324 that may cause the processor 308 to determine a biometric signature from the optical data. For example, the person's eye may be visually unique, and the visual data may be sufficiently unique to provide a biometric signature that may be used to uniquely identify the person. The biometric signature data may be stored as an identifier in a database, for example.

The memory 310 may further include an optical tests module 326 that may cause the processor 308 to send test data to one or more of the computing devices 106. For example, the test data may include objects, object movements, memory testing items, other data, or any combination thereof. In some implementations, the computing device 106(1) may provide the test data to the VR headset 104. The computing device 106(2) may provide the test data to the smart glasses 130. The computing device 106(3) may provide the test data to its display. Other implementations are also possible.

The memory 310 can also include an eye movement analysis module 328 that may cause the processor 308 to determine eye movement data from the optical data. For example, the eye movement analysis module 328 may determine smooth or irregular eye movements. Further, the eye movement analysis module 328 can determine divergence from the optical data. Other examples are also possible.

The memory 310 may further include a facial muscle movement analysis module 330 that may cause the processor 308 to determine muscle movements in the area around the person's eyes. For example, the facial muscle movement analysis module 330 may detect muscle twitches and other muscle movements. In some implementations, such muscle movements may provide insights related to neurological issues or impairments. Other implementations are also possible.

The memory 310 can also include a pupillary reflexes analysis module 332 that may cause the processor 308 to determine changes in the pupillary reflexes from the optical data. For example, exposure to varying intensities of brightness may cause the pupil to dilate or constrict, and pupil reflexes analysis module 332 may determine a rate of change of the pupil size, variations or irregularities in the pupil shape, or other parameters over time, which may be used to assess brain stem function. In some instance, abnormal pupillary reflex may be indicative of optic nerve injury, oculomotor nerve damage, brain stem lesions (such as tumors), and certain medications. The pupillary reflex analysis module 332 may be used to evaluate a person's health independent of any known impact or injury. Other implementations are also possible.

The memory 310 can also include a blood flow analysis module 334 that may cause the processor 308 to determine color variations in a time series of images, which color variations may be imperceptible to the human eye, but which may be indicative of capillary blood flow. For example, as blood flows into the capillary, the color values may change, and as blood flows out of the capillary, the color values may change again. Such changes may indicate the person's pulse and other information related to the person's pulse. Other implementations are also possible.

The memory 310 may also include a motion analysis module 336 that may cause the processor 308 to determine movement data associated with the VR headset 104, the smart glasses 130, or the computing device 106. Such movement data may be indicative of dizziness or loss of balance. Other implementations are also possible.

The memory 310 can further include a pressure analysis module 338 that may cause the processor 308 to determine ocular pressure based on eye movements, such as vibrations or other movements, dimension data, other data, or any combination thereof. For example, the pressure analysis module 338 may detect undulations in a time series of image data. Other implementations are also possible.

The memory 310 may include a memory analysis module 340 that may cause the processor 308 to compare the person's responses to memory data presented to the person 102 to determine whether the responses match. For example, the graphical interface may display information, such as a list of words, a set of objects, or other information, and may instruct the person 102 to memorize the information. Subsequently, the graphical interface may test the recall of the person 102. Short-term memory loss may be indicative of impairment. Other implementations are also possible.

The memory 310 can include a comparison module 342 that may cause the processor 308 to compare data received from the computing device 106 to one or more baselines 344 to determine a deviation from a baseline corresponding to the person 102. For example, the analytics system 102 may retrieve a baseline associated with the person 102 based on biometric data determined by the biometrics module 324. The analytics system 102 may then compare the data to the selected baseline and may determine impairment when the data deviates from the selected baseline by more than a threshold amount. Other implementations are also possible.

In some implementations, the analytics system 302 may receive image data from a computing device 106, perform the image processing analysis to determine impairments, and send data indicative of impairment 120 to the computing device 106. In other implementations, the analytics system 302 may process data received from the computing devices 106 to determine baselines 344 independent of a person 102. In some implementations, the analytics system 302 may process the data over time to determine an average baseline and other data. In some implementations, data from multiple computing devices 106 may be analyzed to determine average baseline data and other parameters that can be used to diagnose neurological impairments and other information. Other implementations are also possible.

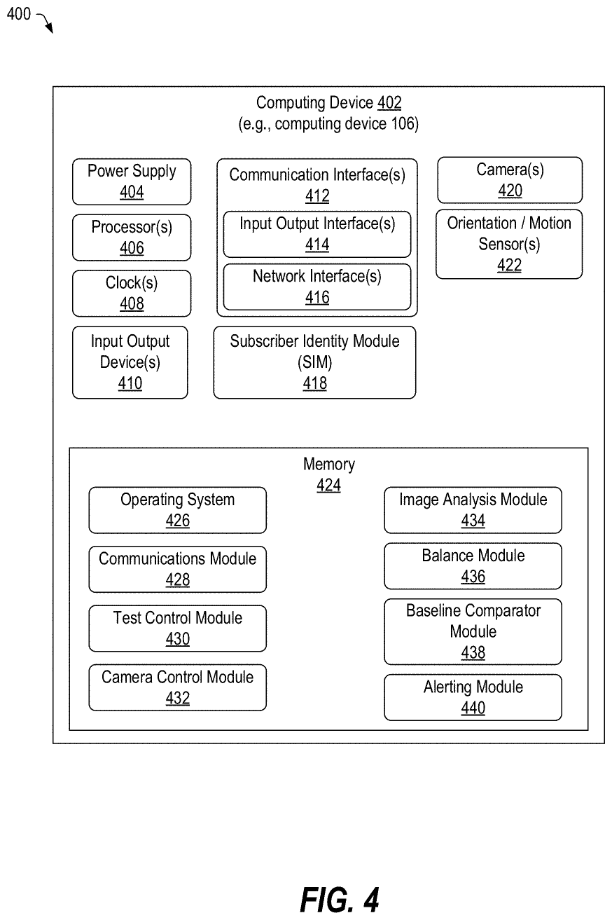

FIG. 4 depicts a block diagram 400 of a computing device 402, in accordance with certain embodiments of the present disclosure. The computing device 402 may be an embodiment of the computing device 106 of FIG. 1. The computing device 402 may be a smartphone, a tablet computer, a laptop computer, another computing device, or any combination thereof.

The computing device 402 may include one or more power supplies 404 to provide electrical power suitable for operating components of the computing device 402. The power supply may include a rechargeable battery, a fuel cell, a photovoltaic cell, power conditioning circuitry, other devices, other circuits, or any combination thereof

The computing device 402 may further include one or more processors 406 to execute stored instructions. The processors 406 may include one or more cores. Further, one or more clocks 408 may provide information indicative of date, time, clock flops, and so on. For example, the processor(s) 406 may use data from the clock 408 to generate a timestamp, to initiate a scheduled action, to correlate image data to data provided to the display, and so on. The computing device 402 may include one or more busses, wire traces, or other internal communications hardware that allows for transfer of data and electrical signals between the various modules and components of the computing device 402.

The computing device 402 may include one or more communications interfaces 412 including input/output (I/O) interfaces 414, network interfaces 416, other interfaces, and so on. The communications interfaces 412 may enable the computing device 402 to communicate with another device, such as the analytics system 302, other computing devices 402, other devices, or any combination thereof through a network 304 via a wired connection or wireless connection. The I/O interfaces 414 may include wireless transceivers as well as wired communication components, such as a serial peripheral interface bus (SPI), a universal serial bus (USB), other components, or any combination thereof.

The I/O interfaces 414 may also couple to one or more I/O devices 410. The I/O devices 410 may include input devices, output devices, or combinations thereof. For example, the I/O devices 410 may include touch sensors, keyboards or keypads, pointer devices (such as a mouse or pointer), microphones, optical sensors (such as cameras), scanners, displays, speakers, haptic devices (such as piezoelectric elements to provide vibrations or impulses), triggers, printers, global positioning devices, other components, or any combination thereof. The global positioning device may include a global positioning satellite (GPS) circuit configured to provide geolocation data to the computing device 402.

The computing device 402 may include a subscriber identity module (SIM) 418. The SIM 418 may be a data storage device that may store information, such as an international mobile subscriber identity (IMSI) number, encryption keys, an integrated circuit card identifier (ICCID), communication service provider identifiers, contact information, other data, or any combination thereof. The SIM 418 may be used by the network interface 416 to communicate with the network 304, such as to establish communication with a cellular or digital communications network.

The computing device 402 may further include one or more cameras 420 or other optical sensor devices, which may capture optical data (images). For example, the cameras 420 may capture image data associated with a user automatically or in response to user input. Further, the computing device 402 may include one or more orientation/motion sensors 422. For example, the orientation/motion sensors 422 may include gyroscopic sensors, accelerometers, tilt sensors, and so on. In some implementations, the orientation/motion sensors 422 may cause the processor 406 to alter the orientation of data presented to a display of the input/output interfaces 414 according to the orientation of the computing device 402. In other implementations, the orientation/motion sensors 422 may generate signals indicative of motion, which may reflect dizziness or imbalance.

The computing device may include one or more memories 424. The memory 424 may include non-transitory computer-readable storage devices, which may include an electronic storage device, a magnetic storage device, an optical storage device, a quantum storage device, a mechanical storage device, a solid-state storage device, other storage devices, or any combination thereof. The memory 424 may store computer-readable instructions, data structures, program modules, and other data for the operation of the computing device 402. Some example modules are shown stored in the memory 424, although, alternatively, the same functionality may be implemented in hardware, firmware, or as a system on a chip.

The memory 424 may include one or more operating system (OS) modules 426, which may be configured to manage hardware resource devices, such as the I/O interfaces 414, the network interfaces 416, the I/O devices 410, and the like. Further, the OS modules 426 may implement various services to applications or modules executing on the processors 406.

The memory 424 may include a communications module 428 to establish communications with one or more other devices using one or more of the communication interfaces 412. For example, the communication module 428 may utilize digital certificates or selected communication protocols to facilitate communications.

The memory 424 may include a test control module 430 to generate visual tests that may be provided to the display or that may be sent to the smart glasses 130 or to the VR headset 104, depending on the implementation. The visual tests may include moving objects, information for memory testing, and other tests. The test control module 430 may control the content, the presentation (including timing), and may initiate operation of the one or more cameras 420 to correspond to presentation of the visual tests.

A camera control module 432 may control operation of the one or more cameras 420 in conjunction with the test control module 430 to capture optical data associated with the person's eyes and face surrounding the eyes. For example, in response to initiation of the visual test, the camera control module 432 may activate the one or more cameras 420 to capture optical data associated with the person. The optical data may include a time series of images of the person's eyes, the facial area that surrounds the eyes of the person, other image data, or any combination thereof that are captured during a period of time that corresponds to the presentation of the visual tests.

The memory 424 may further include an image analysis module 434 to determine parameters associated with the person's eyes and face. The parameters may include eye movement data, pupil reflexes data, pupil shape data, color variation data, facial movement data, eye shape data, blood flow data, and various other parameters. In some implementations, the image analysis module 434 may detect neurological impairment based on the parameters.

The memory 424 may further include a balance module 436 that may utilize orientation and motion data from the orientation/motion sensors 422 to determine balance data associated with the person 102. For example, the balance module 436 may detect an impairment based on changes in the orientation and motion data over time, which may be indicative of dizziness or imbalance. Other implementations are also possible.

A baseline comparator module 438 may retrieve baseline data from the memory 424 or from the analytics system 302 and may compare the parameters associated with the person's eyes and face and the balance data to the baseline data. The baseline data may include one or more baselines associated with the person 102. Alternatively, the baseline data may include an average baseline associated with multiple different persons. Other implementations are also possible.

An alerting module 440 may generate a graphical interface, an email, a text message, or another indicator to notify an operator of the impairment (or lack thereof) of the person. For example, the alerting module 440 may provide a popup notice to the display including data indicative of impairment of the person 102. In another example, the alerting module 440 may send an email or text message to an administrator (such as a high school athletic director or medical personnel) including data indicative of impairment of the person 102. Other implementations are also possible.

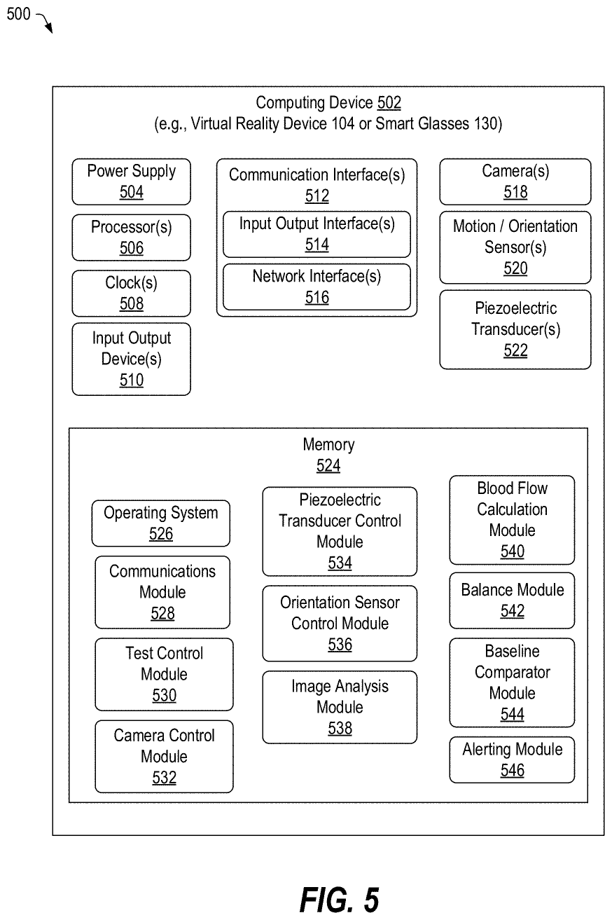

FIG. 5 depicts a block diagram 500 of a computing device 502 such as a VR device 104 or a smart glasses device 130, in accordance with certain embodiments of the present disclosure. The computing device 502 may be an embodiment of the VR device 104 or the smart glasses 130 of FIG. 1.

The computing device 502 may include one or more power supplies 504 to provide electrical power suitable for operating components of the computing device 502. The power supply may include a rechargeable battery, a fuel cell, a photovoltaic cell, power conditioning circuitry, other devices, other circuits, or any combination thereof. For example, the power supply may include a power management circuit configured to receive a power supply via a USB connection to a computing device 106. Other implementations are also possible.

The computing device 502 may further include one or more processors 506 to execute stored instructions. The processors 506 may include one or more cores. Further, one or more clocks 508 may provide information indicative of date, time, clock flops, and so on. For example, the processor(s) 506 may use data from the clock 508 to generate a timestamp, to initiate a scheduled action, to correlate image data to data provided to the display, and so on. The computing device 502 may include one or more busses, wire traces, or other internal communications hardware that allows for transfer of data and electrical signals between the various modules and components of the computing device 502.

The computing device 502 may include one or more communications interfaces 512 including input/output (I/O) interfaces 514, network interfaces 516, other interfaces, and so on. The communications interfaces 512 may enable the computing device 502 to communicate with another device, other computing devices 106, other devices, or any combination thereof through a wired connection or wireless connection 108. The I/O interfaces 514 may include wireless transceivers as well as wired communication components, such as a serial peripheral interface bus (SPI), a universal serial bus (USB), other components, or any combination thereof.

The I/O interfaces 514 may also couple to one or more I/O devices 510. The I/O devices 510 may include input devices, output devices, or combinations thereof. For example, the I/O devices 510 may include touch sensors, pointer devices, microphones, optical sensors (such as cameras), displays, speakers, haptic devices (such as piezoelectric elements to provide vibrations or impulses), other components, or any combination thereof. In some implementations, the I/O devices 510 may include rocker switches, buttons, or other elements accessible by a user to activate and interact with the computing device 502.

The computing device 502 may further include one or more cameras 518 or other optical sensor devices, which may capture optical data (images). For example, the cameras 518 may capture image data associated with a user automatically or in response to user input. Further, the computing device 502 may include one or more orientation/motion sensors 520. For example, the orientation/motion sensors 520 may include gyroscopic sensors, accelerometers, tilt sensors, and so on. In some implementations, the orientation/motion sensors 520 may cause the processor 506 to alter the orientation of data presented to a display of the input/output interfaces 514 according to the orientation of the computing device 502. In other implementations, the orientation/motion sensors 520 may generate signals indicative of motion, which may reflect dizziness or imbalance.

The computing device 502 may include one or more piezoelectric transducers 522. The piezoelectric transducer 522 may be configured to vibrate or generate an impulse in response to electrical signals. For example, the piezoelectric transducer 522 may apply a vibration or pulse to the person's face, and the camera 518 may capture optical data including undulations of the person's skin, facial muscles, eyes, or any combination thereof in response to the vibration or pulse. In some implementations, the rate of decay of the undulations (or the distance traveled from the source) may be indicative of ocular swelling or pressure. Other implementations are also possible.

The computing device 502 may include one or more memories 524. The memory 524 may include non-transitory computer-readable storage devices, which may include an electronic storage device, a magnetic storage device, an optical storage device, a quantum storage device, a mechanical storage device, a solid-state storage device, other storage devices, or any combination thereof. The memory 524 may store computer-readable instructions, data structures, program modules, and other data for the operation of the computing device 502. Some example modules are shown stored in the memory 524, although, alternatively, the same functionality may be implemented in hardware, firmware, or as a system on a chip.

The memory 524 may include one or more operating system (OS) modules 526, which may be configured to manage hardware resource devices, such as the I/O interfaces 514, the network interfaces 516, the I/O devices 510, and the like. Further, the OS modules 526 may implement various services to applications or modules executing on the processors 506.

The memory 524 may include a communications module 528 to establish communications with a computing device 106 using one or more of the communication interfaces 512. For example, the communication module 528 may utilize digital certificates or selected communication protocols to facilitate communications.

The memory 524 may include a test control module 530 to generate or otherwise render visual tests that may be provided to the display. The visual tests may include moving objects, information for memory testing, and other tests. The test control module 530 may control the content, the presentation (including timing), and may initiate operation of the one or more cameras 518 to correspond to presentation of the visual tests.

A camera control module 532 may control operation of the one or more cameras 518 in conjunction with the test control module 530 to capture optical data associated with the person's eyes and face surrounding the eyes. For example, in response to initiation of the visual test, the camera control module 532 may activate the one or more cameras 518 to capture optical data associated with the person. The optical data may include a time series of images of the person's eyes, the facial area that surrounds the eyes of the person, other data, or any combination thereof captured during a period of time that corresponds to the presentation of the visual tests.

The memory 524 may further include a piezoelectric transducer control module 534 to control the piezoelectric transducers 522 to produce the vibrations or impulses. For example, the piezoelectric transducer control module 534 may send an electrical signal to the piezoelectric transducer 522 to initiate a vibration or impulse, which may be applied to the person's face.

An orientation sensor control module 536 may control the orientation sensors 520 to determine orientation and motion changes. For example, as a person 102 moves around while wearing the computing device 502, the orientation or motion data may be generated, which may be indicative of the dizziness or imbalance of the person. Other implementations are also possible.

The memory 524 may include an image analysis module 538 to determine parameters associated with the person's eyes and face. The parameters may include eye movement data, pupil reflexes data, pupil shape data, color variation data, facial movement data, eye shape data, blood flow data, and various other parameters. In some implementations, the image analysis module 538 may detect neurological impairment based on the parameters. Other implementations are also possible.

The memory 524 may further include a blood flow calculation module 540 to determine blood flow to the eyes and the facial area around the eyes based on color changes over time with respect to some of the image data. For example, the blood flow calculation module 540 may measure the person's heart rate and observe blood flow through capillaries in the skin based on color changes over time. Other implementations are also possible.

The memory 524 may also include a balance module 542 that may utilize orientation and motion data from the orientation/motion sensors 520 determined by the orientation sensor control module 536 to determine balance data associated with the person 102. For example, the balance module 542 may detect an impairment based on changes in the orientation and motion data over time, which may be indicative of dizziness or imbalance. Other implementations are also possible.

A baseline comparator module 544 may retrieve baseline data from the memory 524, from a computing device 106, or from the analytics system 302 and may compare the parameters associated with the person's eyes and face and the balance data to the baseline data. The baseline data may include one or more baselines associated with the person 102. Alternatively, the baseline data may include an average baseline associated with multiple different persons. Other implementations are also possible.

An alerting module 546 may generate a graphical interface, an email, a text message, or another indicator to notify an operator of the impairment (or lack thereof) of the person. For example, the alerting module 546 may provide a popup notice to the display including data indicative of impairment of the person 102. In another example, the alerting module 546 may send an email or text message to an administrator (such as a high school athletic director or medical personnel) including data indicative of impairment of the person 102. Other implementations are also possible.

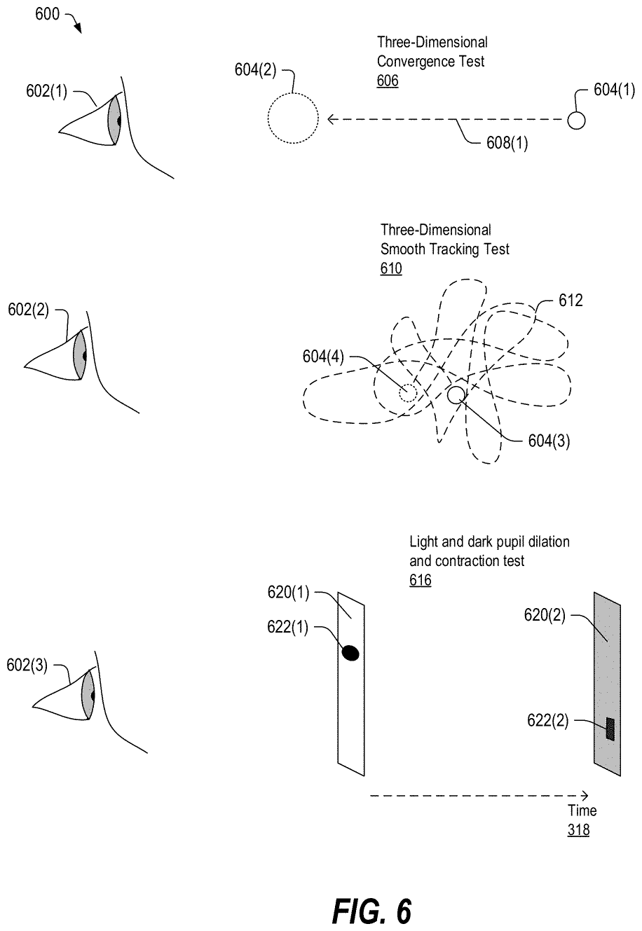

FIG. 6 depicts a diagram 600 of optical test data that can be presented on one of the computing devices of FIGS. 4 and 5, in accordance with certain embodiments of the present disclosure. For example, the optical test data may be presented to a display of the VR headset 104, the smart glasses 130, and the computing device 106.

In the illustrated diagram 600, profiles 602 are shown, which represent the relative position of a pair of eyes being presented with different visual tests, which may be used to cause the eyes to move, the pupils to dilate, and so on. The cameras 518 may capture image data of the eyes 602 and the face of the person 102 as the person observes the visual data.

The person's eyes of the profile 602(1) may be presented with a three-dimensional convergence test 606 in which an object 604 appears to move three-dimensionally toward the person's eyes. In this example, the object 604(1) begins at a distance from the person's eyes and appears to move along the path 608(1), growing larger as the object approaches, as illustrated by the object 604(2). The convergence test 606 causes the object 604 to advance to a point between the person's eyes, while the camera 518 in FIG. 5 or the camera 420 in FIG. 4 captures optical data associated with the person's eyes. The optical data correlated to the position of the object in the convergence test 606 can be used to detect the distance at which the person's eyes diverge. In some implementations, the divergence may provide data indicative of impairment.

The person's eyes of the profile 602(2) may be presented with a three-dimensional smooth tracking test 610 in which an object 604 moves along a path 612 from the object 604(3) to the object 604(4), growing and shrinking along the path to provide an appearance of three-dimensional motion. As the three-dimensional smooth tracking test 610 is provide to the display, the cameras 420 in FIG. 4 or the cameras 518 in FIG. 5 may capture optical data associated with the person's eyes. The optical data correlated to the position of the object in the smooth tracking test 610 can be used to detect irregular or non-smooth movement of the eyes, which may be indicative of impairment.

The person's eyes of the profile 602(3) may be presented with a light and dark pupil reflexes and contraction test 616 in which the position, shape, color, intensity, or other parameters of one or more objects 622(1) and 622(2) may change over time as the background 620 also changes in color, intensity, and so on. In this example, an elliptical shape 622(1) may be presented at a first position and a first time on a first background 620(1) and a second rectangular shape 622(2) may be presented at a second position at a second time and on a second background 620(2). The changing background intensity may be received as changes in light by the pupils, causing the pupils to dilate or contract. As the test 616 is provided to the display, the cameras 420 of FIG. 4 or 518 of FIG. 5 may capture optical data associated with the person's eyes. The optical data correlated to the position of the object 622 in the test 616 together with the changing intensity (brightness) of the background 620 can be used to detect rates of pupil reflexes or contraction and irregular shaped pupils, one or more of which may be indicative of impairment. Other implementations are also possible.

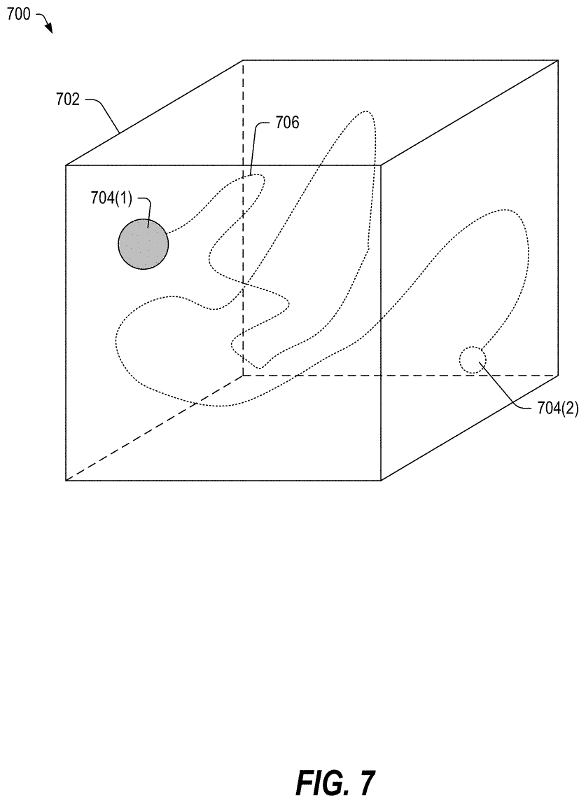

FIG. 7 depicts a diagram of an eye-tracking test 700 that uses three-dimensional movement, in accordance with certain embodiments of the present disclosure. In this example, a three-dimensional space 702 is depicted, which may represent the visual data presented to the display of the VR headset 104, the smart glasses 130, or the computing device 106. The eye-tracking test 700 may depict an object 704 that follows a path 706 within the three-dimensional space 702 changing sizes and color intensity. The object 704(1) may thus have a larger size than the object 704(2), which appears to be further away.

The visual information presented to the display may take a variety of forms. Such forms may include an eye test chart, with letters that get smaller with each row of the eye chart to detect blurry vision. Further, such forms may include moving objects, flashing objects, and so on. Rapid eye response may be tested by presenting objects in various locations and at various distances while the camera 420 in FIG. 4 or 518 in FIG. 5 tracks the person's eye movements. Other implementations are also possible.

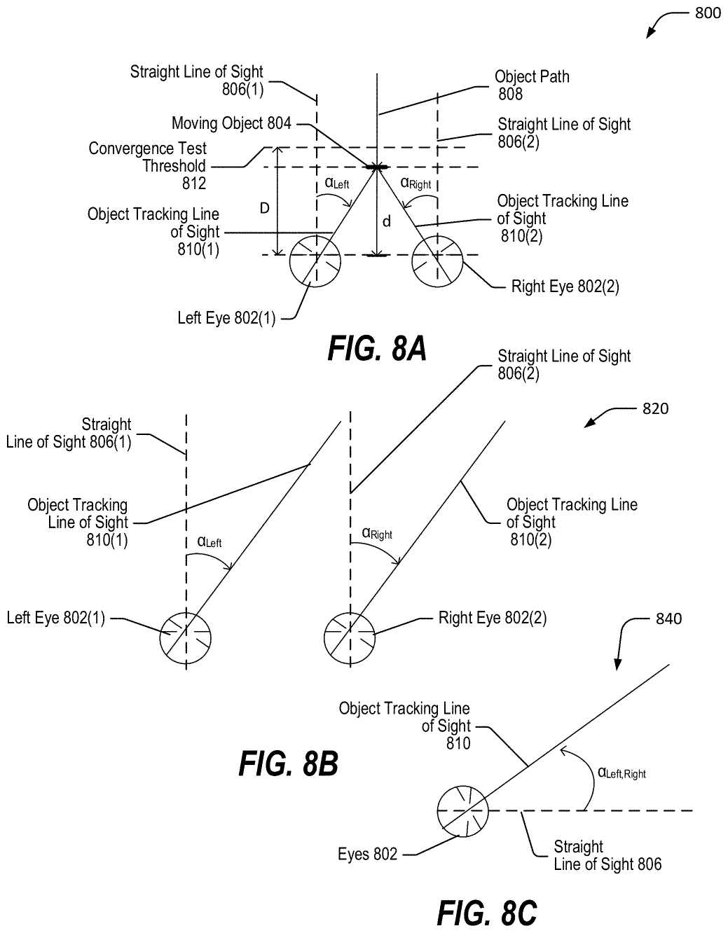

FIGS. 8A-8C depict view angles that may be used to determine impairment, in accordance with certain embodiments of the present disclosure. In FIG. 8A, a view 800 is shown from above the person's head during a 3D convergence test. In this example, the left eye 802(1) and the right eye 802(2) are shown with a straight line of sight 806(1) and 806(2) respectively. The display may present an object 804 that appears to move from a distance away toward a point between the person's eyes 802 along an object path 808 that is perpendicular to the person's face (or to an imaginary line extending between and tangent to both of the eyes 202).

In a convergence test, the user's eyes 802(1) and 802(2) may adjust to follow movement of the object 804, such that the left eye 802(1) and the right eye 802(2) may turn (rotate) toward the object 804 as the object 804 appears to move. In some implementations, when the angles of the eyes 802 diverge, the person may see double (e.g., two objects 804). In some implementations, divergence at a virtual distance of 10 centimeters or more may be indicative of a cognitive impairment. Some persons may have a baseline convergence at a distance that is less than 10 cm, and the baseline distance may be compared to a measured divergence to determine cognitive impairment.

In this example, the eyes 802 are turned toward the object 804 such that object tracking lines of sight 810(1) and 810(2) may vary from straight lines of sight 808(1) and 808(2) by left and right angles (α_{Left} and α_{Right}). The device may determine the angles from optical data of the person's face, which may be captured by one or more optical sensors as the person observes the moving object 804. The device may determine a point at which the object tracking line of sight 810 of one of the eyes 802(1) or 802(2) diverges from the object 804. If that point is at a virtual distance that is greater than 10 centimeters or that differs by more than a threshold amount from a baseline distance, the device may determine cognitive impairment. Other implementations are also possible.

In this example, the near point convergence is a linear distance from the eyes 802 to a location in depth at which the object 804 is reported to be doubled (e.g., the person sees two objects 804). The angles (α) of ocular rotation may be measured from straight ahead of the eyes 802. In an example, the vergence angle may be equal to a difference between the left angle (α_{Left}) and the right angle (α_{Right}).

In FIG. 8B, a view 820 from above the person's head is depicted showing the eyes 802 tracking an object moving to the right. As shown, rapid and smooth eye movements within a horizontal plane may be observed. The angles (α) of eye rotation may be measured from straight ahead of the eyes. The horizontal and vertical eye rotations may be treated separately. In this example, the left and right eye rotation angles (α_{Left} and α_{Right}) are depicted.

In FIG. 8C, a view 840 from a side of the person's head is depicted showing the eyes 802 tracking an object moving up. The angles (α) of vertical eye rotation may be measured from a horizontal plane extending from the eyes (and represented by the straight line of sight 806). The vertical eye rotation angles (α_{Left} and α_{Right}) are depicted.

In some implementations, differences in the left and right rotation angles (FIG. 8A or 8B), differences in the light and right rotation angles (FIG. 8C), or any combination thereof may differ from a predefined threshold. Such differences may be indicative of CI. Alternatively, the rotational angles may be compared to baseline angles, and differences from the baseline may be indicative of CI. Other implementations are also possible.

In some implementations, it may be determined from studying baseline convergence and movement data that a generic baseline may be generated, which may be used to evaluate new persons who may not have their own baseline measurements. Deviations from the generic baseline values may indicate a possible injury or other issues indicative of potential cognitive problems.

In some implementations, optical data of the person's face and eyes, including the ocular rotation angles, may be determined as the person observes a moving object, which may move side-to-side, up-and-down, toward and away from the person's eyes, and so on. The object may be presented on a display of virtual reality goggles, smart glasses, a smartphone, or any combination thereof, and the optical data may be captured as the person observes the moving object. The system or device may determine the various angles, the divergence distance, and other eye and facial parameters based on the optical data. Variations in the angles or other facial parameters relative to a baseline associated with the person (or relative to average parameters determined across a plurality of persons) may be used to evaluate possible cognitive impairment of the person.

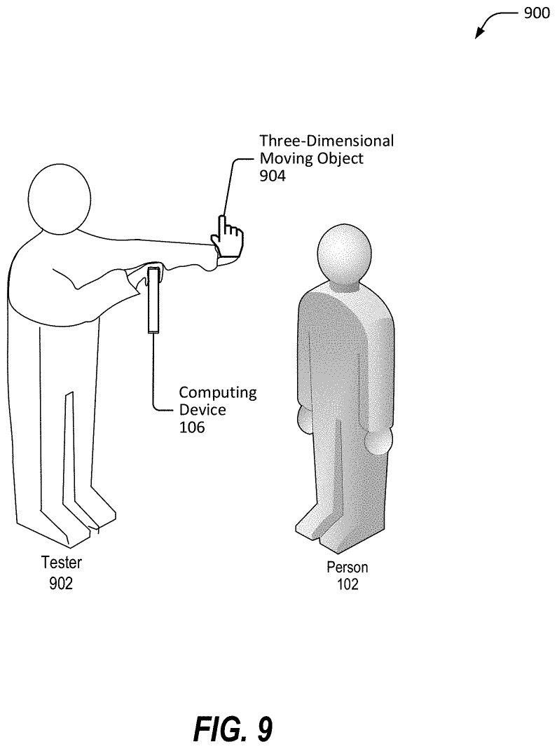

FIG. 9 depicts a system 900 to capture optical data of a person 102 as the person observes a three-dimensional moving object, in accordance with certain embodiments of the present disclosure. In this example, a tester 902, such as a trainer, doctor, or another person, may present a moving object 904. In this example, the moving object 904 may be a finger; however, other moving objects may also be used, such as a pen, a ball, and so on. The tester 902 may move the moving object 904 in three-dimensions in front of the person 902 and may use a computing device 106 to capture optical data associated with the person's eyes as the person 102 observes the moving object 904.

In some implementations, the tester 902 may utilize the computing device 106 to confirm divergence test information, eye movement information, and so on. In this example, the computing device 106 may not present display data for observation by the person 102, but rather may be used as a high-resolution camera to capture the optical data for use in determining whether the person 102 has a cognitive impairment. Other implementations are also possible.

The systems, methods, and devices described herein may be used in a clinical setting, such as in a doctor's office, or may be used in other venues, such as on a sideline at a sporting event. In some implementations, software may be downloaded onto a smartphone and a test may be administered directly by present information on the display of the smartphone while simultaneously capturing optical data of the person's eyes. In other implementations, software may be downloaded onto the smartphone and a first person may move an object around while capturing image data associated with the second person's eyes. In still other implementations, video of the person's eyes may be captured using another device and the video may be uploaded. The system may receive the image data and may process the image data against one or more baselines associated with the person, one or more thresholds, or any combination thereof to determine cognitive impairment. Other implementations are also possible.

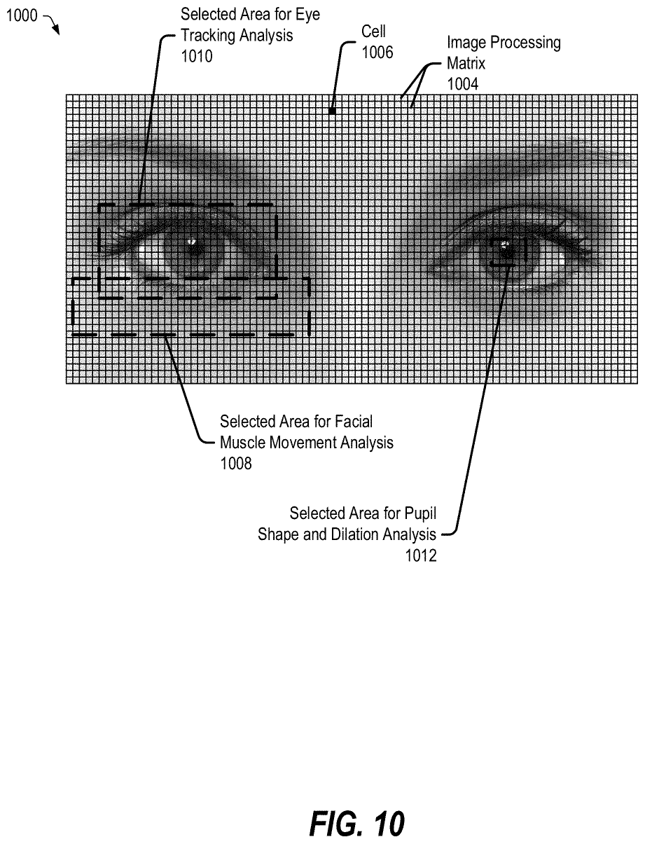

FIG. 10 depicts an image 1000 including an image processing matrix 1004 and including elements or areas for analysis, in accordance with certain embodiments of the present disclosure. The image processing matrix 1004 may divide an image into rows and columns of subset of pixels or image values. Each pixel or image value may represent an intensity in two or more dimensions, such as a red/green/blue (RGB) color spectrum where each pixel has a value within a range of 0-255×0-255×0-255 (or 256×256×256=16,777,216 possible combinations). The number of pixels or image values within each cell 1006 of the matrix 1004 may vary, depending on the implementation.

In this example, subsets of the pixels or image values may be selected for further processing. In this example, a first area 1008 includes a selected subset of pixels or image values for facial muscle movement analysis. A second area 1010 includes a selected subset of pixels or image values for eye tracking analysis. A third area 1012 includes a selected subset of pixels or images values for pupil shape and reflexes analysis.

The captured optical data may include information that is not perceptible to the naked eye, but which may be clearly discerned by the processors. For example, transient color changes that can be detected in the optical data may be imperceptible to human vision, but nevertheless may be used to review information about the person. Such transient color changes may represent blood flowing through capillaries in the eyes and surrounding facial tissue. Further, small tremors in the eye movements may not be perceptible to the naked eye but may represent irregular or non-smooth eye movements. Further, divergence can be accurately determined based on correlations between eye movements and the apparent position of the object presented to the display. In some implementations, the processors may be configured to amplify such small color differences, movements, or other changes to render those difference or changes sufficiently to be seen by a user, such as a physician or trainer. Such amplified differences, movements, or changes may be used to determine one or more conditions of the person. Other implementations are also possible.

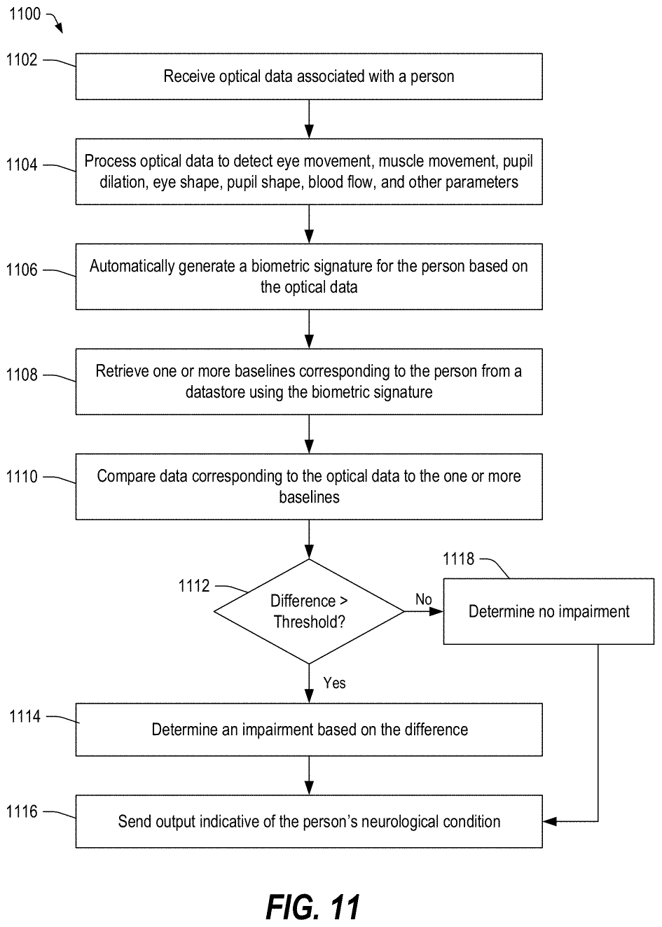

FIG. 11 depicts a flow diagram of a method 1100 of determining impairment based on optical data, in accordance with certain embodiments of the present disclosure. The method 1100 may be implemented on the computing device 116, the analytics system 302, the computing device 402, the computing device 502, or any combination thereof

At 1102, optical data associated with a person is received. The optical data may include images of the person's eyes and facial area surrounding the person's eyes. The optical data may be received from a camera 420, from the VR device 114, or from the smart glasses 130.

At 1104, the optical data may be processed to detect eye movement, muscle movement, pupil reflexes, eye shape, pupil shape, blood flow, and other parameters. For example, the optical data may be processed to detect smooth eye movement while the person's eyes are tracking a moving object, or to detect divergence as an object moves toward a point between the person's eyes. Further, color changes over time may be processed to determine blood flow, and so on.

At 1106, a biometric signature may be automatically generated for the person 102 based on the optical data. The eyes may provide a biometric signature that is unique, at least to the same degree that a fingerprint is considered unique. Accordingly, the optical data may be used to produce a biometric signature that can uniquely identify the person 102.

At 1108, one or more baselines corresponding to the person 102 may be retrieved from a data store using the biometric signature. The one or more baselines may include optical data from previous tests, which may reflect the person's good health or varying degrees of impairment. In an example, a person 102 may be tested when he or she is healthy to produce a healthy baseline. Subsequently, the patent 102 may be tested and the optical data may be compared to the healthy baseline to detect impairment (or to a recent test indicating impairment to determine improvement). Other examples are also possible.

At 1110, data corresponding to the optical data may be compared to one or more baselines. For example, the optical data (or data determined from the optical data) may be compared to a baseline retrieved from a database. Other implementations are also possible.

At 1112, if a difference between the optical data and the baseline is greater than a threshold, impairment may be determined based on the difference, at 1114. It is understood that small variations may exist between tests, and the threshold is used to prevent the small variations from triggering a determination of impairment. Other implementations are also possible.

At 1116, an output indicative of the person's neurological condition is sent. For example, the output may indicate that the person has a neurological impairment, such as a concussion, a chemical impairment, another cause of impairment, or any combination thereof. In some examples, dehydration of the person 102 may also be reflected in the optical data. Other implementations are also possible.

Returning to 1112, if the difference is less than the threshold, no impairment is determined, at 1118. In an example, if the optical data matches or is similar enough to the baseline, the optical data may be indicative of a healthy person. At 1116, an output indicative of the person's brain condition can be sent. In this instance, the output may indicate that the person 102 is healthy. Other implementations are also possible.

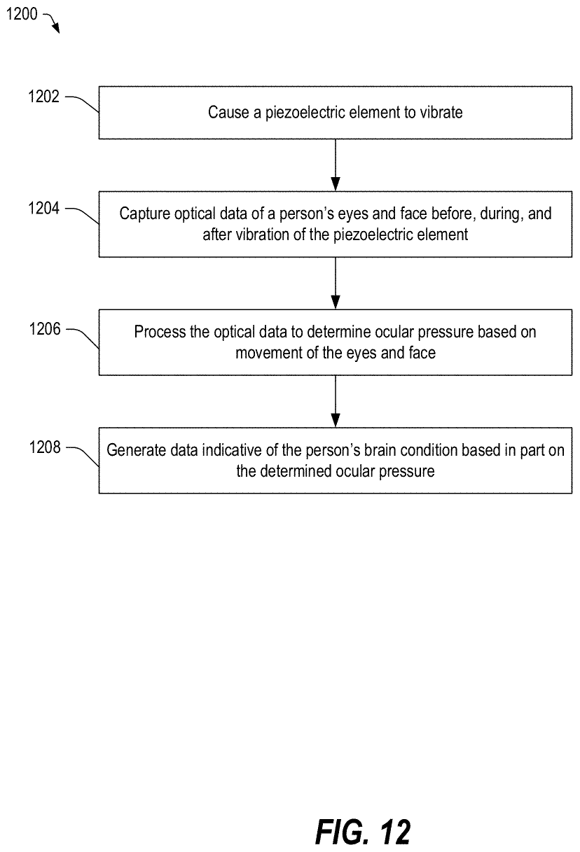

FIG. 12 depicts a flow diagram of a method 1200 of determining impairment based on optically detected ocular pressure, in accordance with certain embodiments of the present disclosure. The method 1200 may be implemented on a system including a VR headset 104 and an associated computing device 106(1) or on smart glasses 130 and an associated computing device 106(2). Other implementations are also possible.

At 1202, a piezoelectric element may be caused to vibrate. For example, a current may be applied to the piezoelectric element to cause vibration or an impulse.

At 1204, optical data of a person's eyes and face may be captured before, during, and after vibration of the piezoelectric element. For example, vibration of the piezoelectric element may cause undulations of the person's facial muscles and eyes, which can be detected in the optical data.

At 1206, the optical data may be processed to determine ocular pressure based on movement of the eyes and face. In one possible implementation, the rate of decay of the undulations may be indicative of ocular pressure, swelling, or other parameters. Other implementations are also possible.

At 1208, data indicative of the person's brain condition or physiological state changes may be generated based in part on the determined ocular pressure. In one example, the data may indicate that the person 102 does not have a concussion. In another example, the data may indicate brain swelling or ocular swelling, which may be indicative of a concussion. Alternatively, the data may be indicative of another condition, such as dehydration, illness, or another condition. Other implementations are also possible.

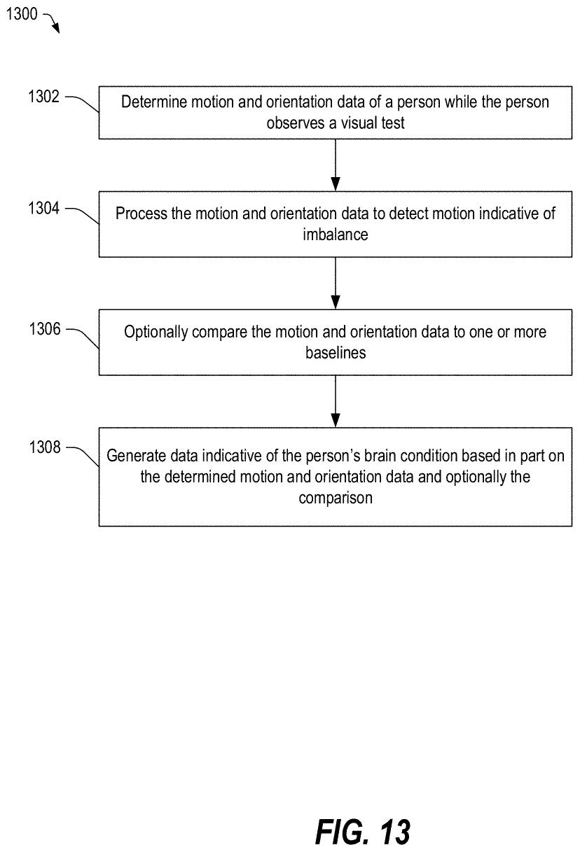

FIG. 13 depicts a flow diagram of a method 1300 of determining impairment based on motion and orientation data, in accordance with certain embodiments of the present disclosure. The method 1300 may be implemented on a system including a VR headset 104 and an associated computing device 106(1), on smart glasses 130 and an associated computing device 106(2), on the computing device 106(3), on the analytics system 302, on the computing device 402, on the computing device 502, or any combination thereof.

At 1302, motion and orientation data of a person 102 may be determined while the person observes a visual test. For example, the motion and orientation data may be determined by motion analysis module 336 of the analytics system 302. In another example, the motion and orientation data may be determined from orientation/motion sensors 422 or from motion/orientation sensors 520.

At 1304, the motion and orientation data may be processed to detect motion indicative of imbalance. For example, relatively rapid changes in motion or orientation may indicate dizziness or imbalance. An unimpaired person 102 may produce motion or orientation data that is substantially stable, while an impaired person 102 may produce time-varying motion or orientation data indicative of instability.

At 1306, the motion and orientation data optionally may be compared to one or more baselines. The baselines may be indicative of prior measurements of the person 102. In an alternative, the baselines may be indicative of average measurements of a plurality of persons 102 over time. Other implementations are also possible.

At 1308, data indicative of the person's brain condition may be generated based, at least in part, on the determined motion and orientation data and optionally the comparison. In some implementations, the data indicative of the person's brain condition (such as a concussion or other impairment) may be determined based on the motion and orientation data by itself, which may indicate that the person's balance is off In other implementations, the motion and orientation data (i.e., the person's movements, tilt angles, and other movement information) may be compared to a baseline associated with the person 102 to determine the person's physiological state changes representative of cognitive impairment. In still other implementations, the motion and orientation data may be compared to a baseline that may represent an average determined from the motion and orientation data from a plurality of persons. Other implementations are also possible.

In conjunction with the systems, methods, and devices of FIGS. 1-13, visual data may be presented to a display for viewing by a person, and optical sensors (such as a camera) may product optical data associated with the person's eyes and facial area surrounding the eyes. The optical data may be processed to determine a neurological impairment. In some implementations, data indicative of impairment may be sent to a computing device.

In some implementations, sensors including optical sensors, pressure sensors, temperature sensors, or other sensors may provide signals that may be processed to determine various parameters associated with the person. Such parameters may be compared to threshold or may be compared to baselines associated with the person to determine deviations that may be indicative of traumatic brain injury or cognitive impairment.

Although the present invention has been described with reference to preferred embodiments, workers skilled in the art will recognize that changes may be made in form and detail without departing from the scope of the invention.

What is claimed is:

1. A system comprising:
a computing device comprising:
one or more sensors to capture data associated with a person's eyes as the person observes one or more objects moving in a three-dimensional space; and
a display to present information related to the captured data.

2. The system of claim 1, wherein the computing device further comprises a processor coupled to the one or more sensors and to the display, the processor to:
compare the captured data to one or more baselines associated with the person to determine one or more differences; and
determine cognitive impairment of the person based on the one or more differences.

3. The system of claim 1, wherein the computing device comprises a processor coupled to the one or more sensors and to the display, the processor to:
compare the captured data to one or more thresholds to determine one or more differences; and
determine cognitive impairment of the person based on the one or more differences.

4. The system of claim 1, wherein the computing device comprises a processor to generate an alert when the captured data is indicative of cognitive impairment.

5. The system of claim 1, wherein the display is configured to present visual information including the one or more objects.

6. The system of claim 1, wherein the computing device comprises a processor coupled to the one or more sensors and to the display, the processor to control the display to present visual information including a convergence test and to determine divergence of one or more of the person's eyes as the person observes the one or more objects moving in the three-dimensional space.

7. The system of claim 6, wherein the processor determines cognitive impairment when a distance at which divergence is determined is greater than one or more of a threshold distance and a baseline distance associated with the person.

8. The system of claim 1, wherein the captured data includes one or more of involuntary eye movement data associated with the patient's eyes, rapid eye movement data associated with the patient's eyes, smooth pursuit data associated with the patient's eyes, or pupil reflexes data associated with the patient's eyes.

9. The system of claim 1, wherein the computing device comprises at least one of smart glasses, a virtual reality headset, an augmented reality headset, a smartphone, and a tablet computer.

10. A system comprises:
a device comprising:
a communications interface to couple to one of a network and a computing device;
a display to present visual information including one or more objects moving in a three-dimensional space;
one or more sensors to capture data associated with a person's eyes as the person observes the one or more objects; and
a processor coupled to the communications interface, the display, and the one or more sensors, the processor to compare the captured data to one or more thresholds or to one or more baselines associated with the person to determine a difference, the processor to provide information related to the comparison to one or more of the display or the computing device

11. The system of claim 10, further comprising:
a computing device comprising:
an interface to receive the optical data from the device;
a processor; and
a memory to store data and to store processor-readable instructions that cause the processor to:
compare the received optical date to one or more of a baseline associated with the person or a threshold;
determine cognitive impairment of the person based on the comparison; and
generate an alert indicative of cognitive impairment.

12. The system of claim 10, wherein the device comprises at least one of smart glasses, a virtual reality headset, an augmented reality headset, a smartphone, and a tablet computer.

13. The system of claim 10, wherein the processor:
determines a baseline corresponding to the patient; and
generates comparative data from one or more repeat tests relative to the determined baseline.

14. The system of claim 10, wherein:
the visual information includes a convergence test that includes a visual representation of an object that appears to move from a distance toward a point that is between the person's eyes; and
wherein the processor generates the data indicative of a distance of the object when a first ocular angle associated with a first eye diverges from a second ocular angle associated with a second eye of the person's eyes.

15. The system of claim 14, wherein the processor determines the impairment when the distance is greater than one or more of a threshold distance and a baseline distance associated with the person.

16. A system comprising:
a computing device comprising:
one or more sensors to capture data associated with a person's eyes as the person observes one or more objects moving in a three-dimensional space; and
a processor coupled to the one or more sensors and configured to generate information related to the capture data; and
a display coupled to the processor and configured to present the generated information.

17. The system of claim 16, further comprising:
a communications interface coupled to the processor and configured to communicate with a data store through one or more of a communications network or a communications link; and
wherein the processor:
determines a baseline corresponding to the patient from a data store; and
generates comparative data from the captured data and the baseline; and
determines the generated information based on the comparative data.

18. The system of claim 16, wherein:
the visual information includes a convergence test that includes a visual representation of an object of the one or more objects that appears to move from a distance toward a point that is directly between the person's eyes; and
wherein the processor determines the generated information based on the distance of the object when a first ocular angle associated with a first eye diverges from a second ocular angle associated with a second eye of the person's eyes.

19. The system of claim 18, wherein the processor determines the cognitive impairment when the virtual distance is greater than one or more of a threshold distance and a baseline distance associated with the patient.

20. The system of claim 16, wherein the captured data includes rapid eye movement data, smooth pursuit data, pupil reflexes data, convergence data, divergence data, facial muscle movement data, blood flow data, eye shape data, and pupil data.
